(19) United States
(12) Patent Application Publication
(10) Pub. No.: US 2017/0071788 A1
(43) Pub. Date: Mar. 16, 2017

(54) CURVED VITRECTOMY PROBE

(71) Applicant: Novartis AG, Basel (CH)

(72) Inventors: Joshua Anderson, Keller, TX (US); Paul R. Hallen, Colleyville, TX (US)

(21) Appl. No.: 15/262,255

(22) Filed: Sep. 12, 2016

Related U.S. Application Data
(60) Provisional application No. 62/218,900, filed on Sep. 15, 2015.

(51) Int. Cl. A61F 9/007; A61B 17/00

(52) U.S. Cl. CPC A61F 9/00736; A61B 17/00234; A61B 2217/005; A61B 2017/00318

(57) ABSTRACT

A vitrectomy probe includes a hand-graspable body and an outer tube extending from the hand-graspable body. The outer tube includes a closed distal end and a first port sized to receive tissue. The vitrectomy probe also includes an inner tube within the outer tube. In various embodiments, the outer tube and/or the inner tube are curved. In some embodiments, the curvature of the outer tube is adjustable.

### DESCRIPTION OF THE RELATED ART

Microsurgical procedures frequently require precision cutting and/or removing of various body tissues. For example, certain ophthalmic surgical procedures require cutting and removing portions of the vitreous humor, a transparent jelly-like material that fills the posterior segment of the eye. The vitreous humor, or vitreous, is composed of numerous microscopic fibrils that are often attached to the retina. Therefore, cutting and removing the vitreous must be done with great care to avoid traction on the retina, the separation of the retina from the choroid, a retinal tear, or, in the worst case, cutting and removal of the retina itself. In particular, delicate operations such as mobile tissue management (e.g. cutting and removal of vitreous near a detached portion of the retina or a retinal tear), vitreous base dissection, and cutting and removal of membranes are particularly difficult.

Microsurgical cutting probes used in posterior segment ophthalmic surgery may include a straight hollow outer cutting member, a straight (or slightly bent for shearing) hollow inner cutting member arranged coaxially with and movably disposed within the hollow outer cutting member, a port extending radially through the outer cutting member near the distal end of the outer cutting member, and a port extending radially through the inner cutting member near the distal end of the inner cutting member (and/or the cutting member may be open at the distal end). Vitreous humor and/or membranes are aspirated into the open port of the outer cutting member and the inner member is actuated to distally extend the inner cutting member. As the inner cutting member extends distally, cutting surfaces on both the inner and outer cutting members cooperate to cut the vitreous and/or membranes, and the cut tissue is then aspirated away through the inner cutting member. Vitreous and/or membranes are then aspirated into the open ports of both the outer and inner cutting members and the inner member is actuated to proximally retract the inner cutting member. The inner and outer cutting members cooperate to again cut vitreous and/or membranes and aspirate the cut tissue away. The actuated extension and retraction of the inner cutting member is repeated at dynamic cycle rates between several tens to several hundred times per second.

### SUMMARY

A vitrectomy probe includes a hand-graspable body and an outer tube extending from the hand-graspable body. The outer tube includes a closed distal end and a first port sized to receive tissue. The vitrectomy probe also includes an inner tube within the outer tube. In various embodiments, the outer tube and/or the inner tube are curved. In some embodiments, the curvature of the outer tube/inner tube is adjustable.

### BRIEF DESCRIPTION OF THE DRAWINGS

For a more complete understanding of the present invention, reference is made to the following description taken in conjunction with the accompanying drawings in which:

FIG. 1 is an illustration of an exemplary surgical system according to one aspect of the present disclosure consistent with the principles and teachings described herein.

FIG. 2 is a box diagram of aspects of the exemplary surgical system of FIG. 1 according to an aspect described herein.

FIG. 3 is an illustration of an exemplary vitrectomy probe in cross-section operable in accordance with the principles and teachings described herein.

FIG. 4 is an illustration of an exemplary distal end of the vitrectomy probe in partial cross-section consistent with the principles and teachings described herein.

FIG. 5 is an illustration of an exemplary inner cutting tube consistent with the principles and teachings described herein.

FIGS. 6a-b illustrate an exemplary vitrectomy probe with an adjustable curvature in cross-section in accordance with the principles and teachings described herein.

FIGS. 7a-b illustrate another exemplary vitrectomy probe with an adjustable curvature in cross-section in accordance with the principles and teachings described herein.

FIG. 8 is an illustration of an exemplary method of operation of the vitrectomy probe consistent with the principles and teachings described herein.

It is to be understood that both the foregoing general description and the following detailed description are exemplary and explanatory only and are intended to provide a further explanation of the present invention as claimed.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described systems, devices, and methods, and any further application of the principles of the present disclosure are fully contemplated as would normally occur to one skilled in the art to which the disclosure relates. In particular, it is fully contemplated that the systems, devices, and/or methods described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

The present disclosure is directed to surgical devices, systems, and methods for performing ophthalmic surgeries. The devices, systems, and methods are arranged and configured to provide a curved vitrectomy probe for a vitrectomy procedure. A curved vitrectomy probe may provide access for greater peripheral vitreous removal and potentially facilitate reduced reoperation rates. Potentially, the result may be better patient visual acuity outcomes. Various aspects of these features will be discussed further below.

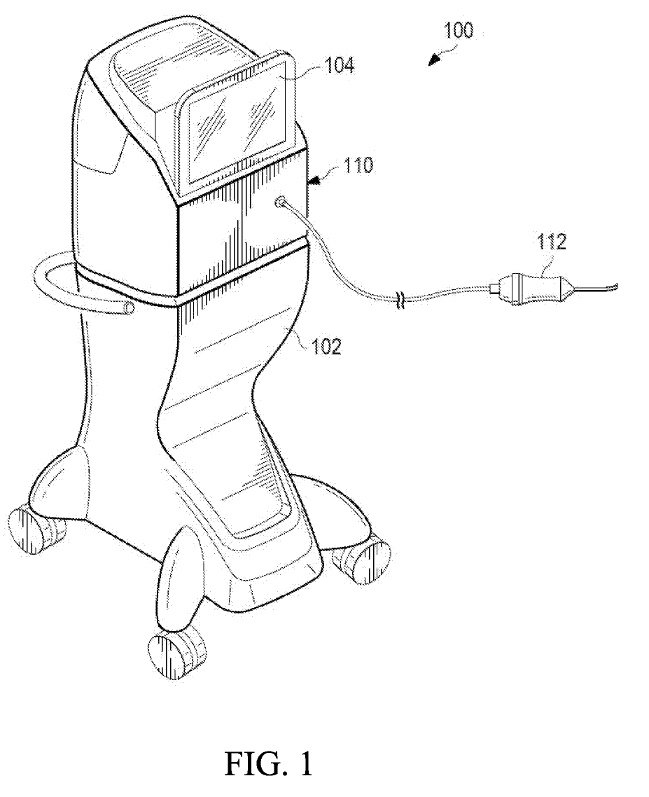

FIG. 1 illustrates a vitrectomy surgical system, generally designated 100, according to an exemplary embodiment. The surgical system 100 includes a base housing 102 and an associated display screen 104 showing data relating to system operation and performance during a vitrectomy surgical procedure. In an embodiment, the base housing 102 may be mobile, for example including wheels to facilitate movement as necessary. In an alternative embodiment, the base housing 102 may not include wheels. The surgical system 100 includes a vitrectomy probe system 110 that includes a vitrectomy probe 112, as will be discussed in more detail below with respect to subsequent figures.

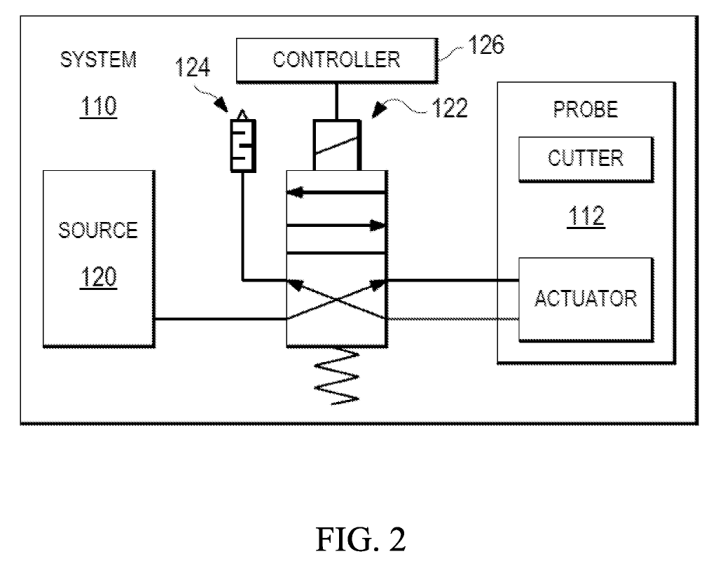

FIG. 2 is a schematic of exemplary components of the vitrectomy probe system 110. The probe system 110 includes the vitrectomy probe 112, a pneumatic pressure source 120, a probe driver shown as an adjustable directional on-off pneumatic driver 122, a muffler 124, and a controller 126. In an embodiment, the controller 126 may be a processor that includes one or more processing cores capable of performing parallel or sequential operations. Alternatively, the controller 126 may be a dedicated piece of hardware such as an application specific integrated circuit (ASIC), to name just a few examples. The source 120, the driver 122, the muffler 124, and the probe 112 are in fluid communication with each other along lines representing flow paths or flow lines. The controller 126 is in electrical communication with the driver 122. In an embodiment, the controller 126 controls operation of both the driver 122 and various aspects of the probe 112, including the frequency of oscillation by way of the actuator as well as a flow rate of fluid to/from the surgical site.

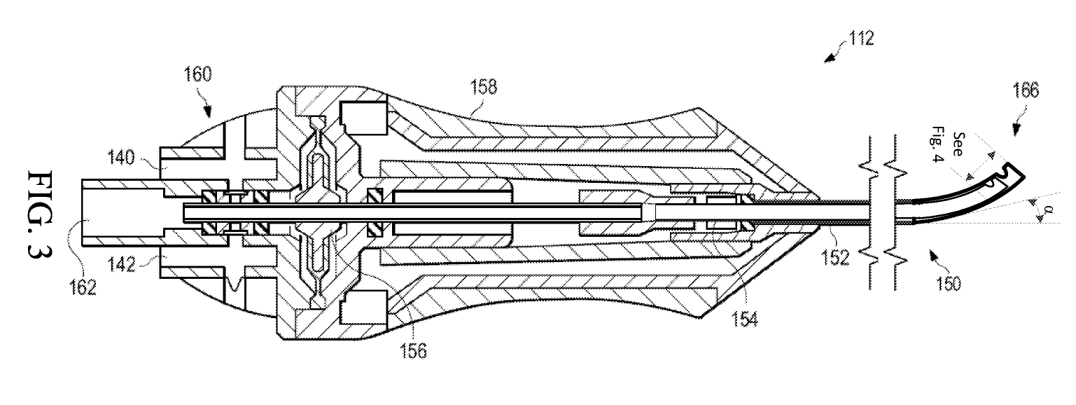

FIG. 3 shows a partial cross-sectional illustration of an exemplary vitrectomy probe, for example the vitrectomy probe 112 introduced in FIGS. 1 and 2. In this example, the vitrectomy probe 112 is a pneumatically driven probe that operates by receiving pneumatic pressure alternating through first and second ports 140 and 142. The probe 112 includes as its basic components a cutter 150 comprising an outer cutting tube 152, an inner cutting tube 154 shown in a non-sectional side view, and a probe actuator or motor shown here as a reciprocating air driven diaphragm 156, all at least partially encased by a housing 158. The housing 158 includes an end piece 160 at the probe proximal end with the first and second air supply ports 140, 142 and one suction port 162 to provide aspiration of materials from the cutter 150.

In an embodiment, the vitrectomy probe system's pneumatic driver 122 (FIG. 2) may be a standard four-way on-off valve. The pneumatic driver 122 may have a solenoid that operates to move the driver to one of the two on-off positions depicted in the example of FIG. 2. Here, the pneumatic driver 122 is in a position to provide pneumatic pressure to the first port 140 (FIG. 3), and to vent pneumatic pressure from the second port 142 (FIG. 3). In this position, pneumatic pressure may pass from the pressure source 120, through the on-off pneumatic driver 122, and to the first port 140 where the pneumatic pressure provides pneumatic power to the vitrectomy probe 112. At the same time, pneumatic pressure at the second port 142 may pass through the on-off pneumatic driver 122 to the muffler 124 where it is exhausted, for example, to the atmosphere. In the other position, the on-off pneumatic driver 122 allows pneumatic pressure to pass from the pressure source 120 to the second port 142, where the pneumatic pressure provides pneumatic power to the vitrectomy probe 112. At the same time, pneumatic pressure at the first port 140 can vent through the on-off pneumatic driver 122 to the muffler 124 where it is exhausted to the atmosphere. The on-off pneumatic driver may be configured to receive operating signals from the controller 126.

In operation, pneumatic pressure is directed alternately from the source 120 to the first and second ports 140, 142 to operate the vitrectomy probe 112. The on-off pneumatic driver 122 alternates between its two positions very rapidly to alternatingly provide pneumatic pressure to the first and second ports 140, 142. Although shown with a single pneumatic driver 122, other embodiments include two pneumatic drivers, one associated with each of the two ports 140, 142. These embodiments operate similar to the manner described, with the drivers being configured to independently receive operating signals from the controller 126 (FIG. 2). Yet other arrangements are contemplated.

Generally, the inner cutting tube 154 oscillates within the outer cutting tube 152 in response to the probe actuator. In an embodiment, the inner cutting tube 154 is driven by air pressure directed on opposing sides of the diaphragm 156. In one example of operation, if air pressure is increased at the first port 140, the diaphragm 156 will move distally, displacing the inner cutting tube 154 relative to the outer cutting tube 152, thereby moving a first cutting edge on a distal end of the inner cutting tube 154 in the distal direction and cutting tissue. This cuts any vitreous material which may have been aspirated into a tissue-receiving outer port of the outer cutting tube 152. The vitreous may be aspirated away at a distal end of the inner cutting tube 154. Venting the pressure at the first port 140 and increasing the pressure at the second port 142 moves the diaphragm 156 proximally which results in moving the inner cutting tube 154 in the proximal direction. In some embodiments, the inner cutting tube 154 may also include a side port for cutting. In this embodiment, as the diaphragm 156 moves proximally, a second cutting edge facing a proximal direction near the distal end of the inner cutting tube 154 may also move in the proximal direction, cutting any vitreous material which may have entered the ports of the inner cutting tube 154 and outer cutting tube 152 while at least partially aligned.

In alternative embodiments, the probe actuator may include a piston motor in place of a diaphragm. In this type of embodiment, the cutter 150 is arranged so that movement of the piston also moves the inner cutting tube 154 of the cutter 150. Yet other embodiments include other types of pneumatic or electric motors that drive the inner cutting tube 154, as will be recognized by those skilled in the relevant art(s).

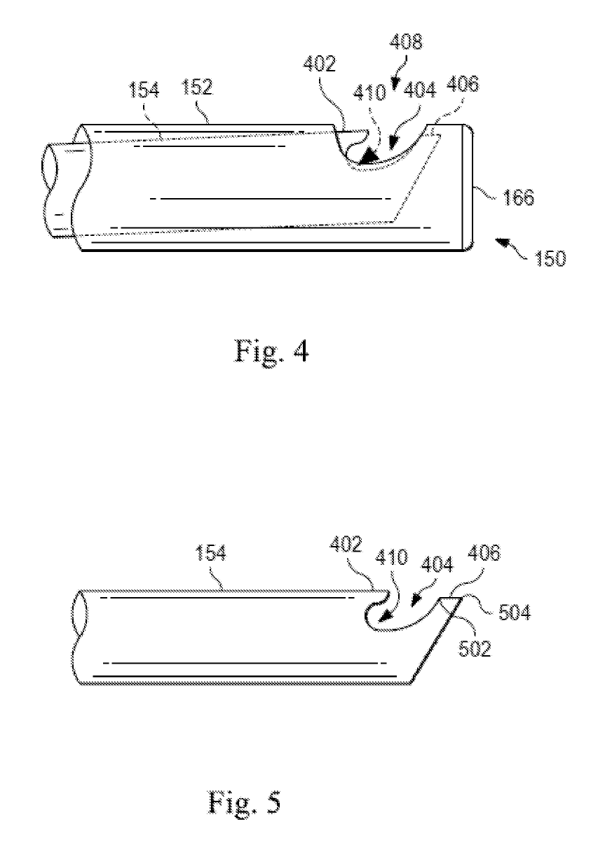

FIG. 4 illustrates an exemplary distal end of the vitrectomy probe 112. FIG. 4 is shown as a straight distal-most section of tube 152, but in some embodiments, the distal-most portion of tube 152 may be curved. FIG. 4 illustrates in more detail the cutter 150 of FIG. 3 showing the outer cutting tube 152 and showing the inner cutting tube 154 in place in the outer cutting tube 152. The cutter 150 includes the inner cutting tube 154 and the outer cutting tube 152. The inner cutting tube 154 fits within the outer cutting tube 152 in a coaxial manner, and the inner cutting tube 154 is axially moveable relative to the outer cutting tube.

The outer cutting tube 152 has a closed end at the distal end 166 and an outer port 408 that may receive various material, such as tissue. In an embodiment, the tissue may be ophthalmic tissue such as vitreous and/or membrane. The outer port 408 has a distal portion, nearest the distal end 166, and a proximate portion. Each of the distal and proximate portions of the outer port 408 may include a cutting edge.

FIG. 5 illustrates a distal portion of the inner cutting tube 154 specifically. FIG. 5 is shown as a straight distal-most section of inner tube 154, but in some embodiments, the distal-most portion of tube 154 may be curved. The inner cutting tube 154 may have an open end at the distal end 166 and an inner port 404 to similarly receive material such as tissue. The inner port 404 has a distal side 502 that forms the proximate side of tip 406. Tip 406 also has a distal side 504 facing the distal end 166 of the cutter 150. The distal and proximate sides 504, 502 of the tip 406 may include cutting edges which, when moving with respect to the cutting edges on the outer port 408, cooperate to cut material such as tissue as will be described further below.

In some embodiments, the inner tip 406 may be curved slightly more than the outer tube to provide bias toward the outer port 408. This different cross section at the tip 406 may provide shearing edges between the inner cutting tube 154 and the outer cutting tube 152. This may facilitate smooth, progressive shearing of vitreous/membranes and may reduce wear to the vitrectomy probe 112 over time. In some embodiments, both the inner and outer tubes may have round cross sections. In alternate embodiments, the slightly increased curve in the tip 406 may impart a unique tubular cross section to the inner cutting tube 154, such as a substantially oval cross section, in contrast to the rest of the inner cutting tube 154 that is approximately round. This may allow an oval cross section at the tip 406 to distribute the sliding wear at the tip 406 over a greater surface area that comes in contact with the inner surface of the distal end of the outer cutting tube 152. The oval cross section of the tip 406 may also decrease the size of the annular space between the tip 406 and the inner surface of the distal end of the outer cutting tube 152, which may decrease the potential for vitreous incarceration and vitreoretinal traction.

Returning to FIG. 5, in some embodiments, the inner port 404 of inner cutting tube 154 has a proximate portion where guiding surface 402 is located. A side lobe 410 may be located on each side of the guiding surface 402 as contours along the periphery of the inner port 404. In some embodiments, the inner cutter may have a simple inner port 404 without side lobes. In some embodiments, the inner cutter may not have an inner port at all (e.g., tissue may be cut primarily by edge 504 traversing outer port 408).

Returning to FIG. 3, the cutter 150 extends from the housing 158 and includes a distal end 166. The outer cutting tube 152 and the inner cutting tube 154 may both be curved cylindrical tubes with a hollow bore. For example, in some embodiments, a curved and elastic inner cannula 154 may be used with a similarly curved and rigid outer tubular cannula 152 to enable precise pneumatic cutting with a curved vitrectomy probe. In one embodiment, a highly elastic inner tube 154 (e.g., made of a superelastic material such as Nitinol) travels co-axially within a curved rigid outer tube 152 (e.g., made of stainless steel or another relatively rigid material) to provide vitreous cutting and removal. In some embodiments, the inner tube 154 may be formed from a nickel titanium alloy, such as Nitinol, which may exhibit superelastic and shape memory properties. Because the inner tube 154 may be superelastic (which term is intended herein as a synonym for the somewhat more technically precise term “pseudoelastic”), the inner tube 154 may be able to withstand a significant amount of deformation when a load is applied and return to its original shape when the load is removed. (Those skilled in the art will appreciate that this property is distinct from, although related to, “shape memory”, which refers to a property exhibited by some materials in which an object that is deformed while below the material's transformation temperature returns to its former shape when warmed to above the transformation temperature. Nitinol exhibits both properties; superelasticity is exhibited above the transformation temperature.) Other superelastic (or other flexible/elastic) materials may also be used to form the inner cutter.

As seen in FIG. 3, the degree a of curve may be approximately in a range of 1 degree to 180 degrees. In these embodiments, the inner cutter may have a similar curvature to the outer tube 152. In some embodiments, the outer tube 152 and inner tube may be substantially straight except for a curve at a distal end portion of the outer tube 152 and inner tube 154. For example, the curvature may be along the distal most 5%, 10%, 15%, 20%, 25%, 30%, etc. length of the shaft 152 that extends out of the housing 158. For example, the outer tube 152 may be curved along 5to 10 percent of a distal most length of the outer tube 152 external to the hand-graspable body (or 11 to 20 percent or 21 to 30 percent). In alternate embodiments, the curvature may start on a proximal end of the shaft 152 and extend along most of the shaft 152. The curve may extend in an upward direction, such that if the probe is being held in an operative fashion, the shaft extends away from the ground. In some embodiments, the curve may extend in a downward direction, such that if the probe is being held in an operative fashion, the shaft extends toward the ground. Other embodiments are also contemplated (e.g., curved in a sideways fashion).

In another embodiment, the device could be composed of a curved and elastic inner cannula and a similarly curved and elastic outer tubular cannula. In these embodiments, both the inner and outer cannula may be constructed from a superelastic material such as Nitinol (or different flexible/elastic materials).

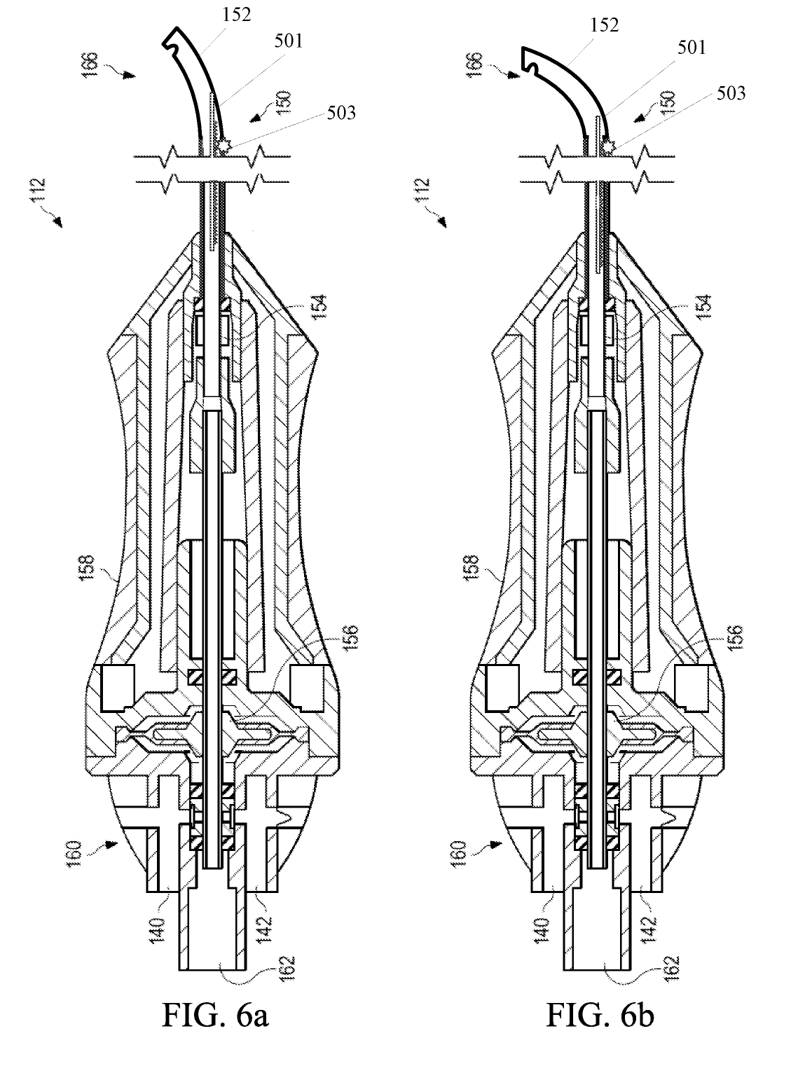

In various embodiments, a mechanism and actuator could be utilized to make an articulating device vitrectomy probe. For example, a user actuated slider, lever, or wheel (e.g., actuated through movement by the user's fingers) could be used to straighten or curve the distal shaft on demand. As seen in FIGS. 6a-b, a wheel gear 503 may be turned to engage a toothed shaft 501 to move the shaft 501 up and down inside the outer tube 152. The outer tube 152 may straighten as the toothed shaft engages an inner surface of the outer tube 152 to oppose the curvature (as seen in FIG. 6a). As the toothed shaft is withdrawn, the outer tube 152 may return to a more curved shape (as seen in FIG. 6b). In some embodiments, the wheel gear 503 could be rotated, for example, externally by a thumb. In some embodiments, the wheel gear 503 may be rotated by an electro/mechanical actuator (e.g., actuated by an external switch or by a signal from a surgical console). Other actuators are also contemplated.

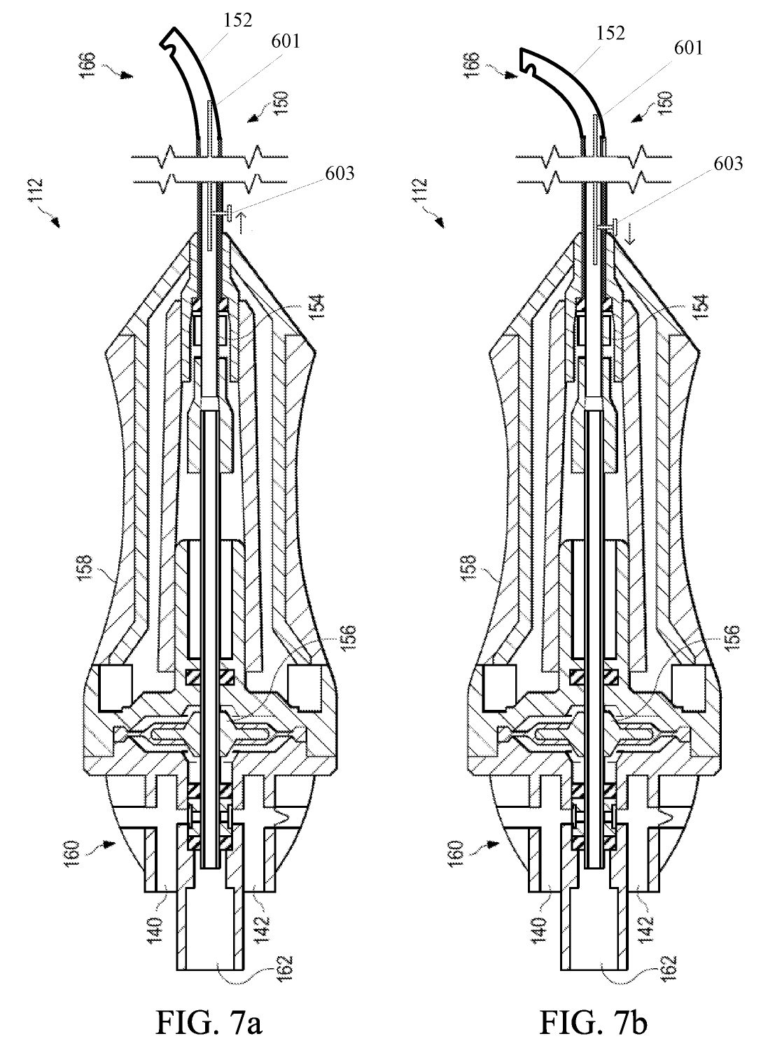

As another example, a slider 603 may be used to slide an internal shaft 601 up and down (as seen in FIGS. 7a-b). The slider 603 may slide along a slot in the side wall of the outer tube 152. In some embodiments, the slider 603 may be frictionally engaged in the slot such that the slider 603 holds position as the slider is moved up and down the slot. As seen in FIG. 6a, moving the slider 603 toward a distal end of the outer tube 152 may move an internal shaft 601 along the outer tube 152 to straighten the outer tube 152. As the slider 603 is withdrawn, the outer tube 152 may return to a more curved shape (as seen in FIG. 7b). (Note, in FIGS. 6a-7b, the inner tube 154 is not shown to show the toothed shaft 501 and shaft 601 more clearly). Also, while the shaft 501 and 601 are shown toward the middle of the outer tube 152 for clarity, the shafts 501 and 601 may abut a side wall of the outer tube 152 between the clearance of the outer tube 152 and inner tube 154. Further, the wheel gear 503 and slider 603 may be placed at other locations along the outer tube 152 or even along the body of the vitrectomy probe housing 158.

The curvature of the outer tube 152 may be adjustable during surgery or may be adjusted prior to surgery and hold the constant set curvature during surgery. In some embodiments, the curved probe may provide better access to the far peripheral vitreous, while simultaneously providing clearance around the human crystalline lens, which, if touched, may precipitate cataract formation.

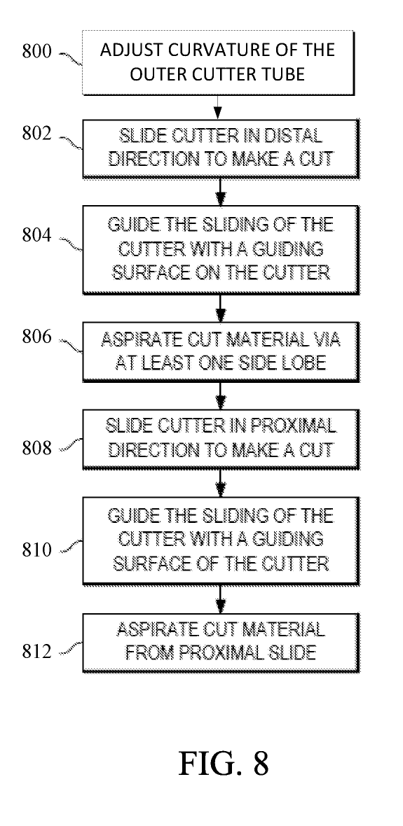

FIG. 8 is an illustration of an exemplary method of operation of a vitrectomy probe. In an embodiment, the vitrectomy probe 112 may be operating as part of the vitrectomy probe system 110 discussed above. In operation, the distal end 166 of the vitrectomy cutter 150 may be introduced into an eye of a patient to be treated for an ocular condition. A vacuum may be used to draw fluid and tissue, such as vitreous, into the outer port during operation.

At 800, the curvature of the outer tube 152 of the vitrectomy probe may be adjusted (e.g., as discussed above with respect to FIGS. 6a-7b). In some embodiments, the curvature of the outer tube may be locked prior to the start of surgery. In some embodiments, the curvature may not be locked (e.g., the curvature may be adjustable during surgery). In some embodiments, the amount of curvature may be indicated on the probe. For example, if the probe has a fixed curvature, the angle a of curvature may be printed on the vitrectomy probe. In some embodiments, different α's of curvature may be indicated near the mechanism for adjusting the curvature of the probe (e.g., next to the slot on the slider embodiment) to indicate the curvature of the probe corresponding to the position of the adjuster.

At 802, the vitrectomy probe 112 causes the inner cutting tube 154 to slide in a distal direction. As the cutting edge at the tip of the inner cutting tube 154 moves distally, the cutting edge cuts tissue that has entered the outer port of the outer cutting tube 152. The inner cutting tube 154 may be curved/bent slightly more than the outer tube near its distal end to provide a flexural side load, causing the guiding surface to be engaged with an inner surface of the outer cutting tube 152. In this angled position, the tip is disposed in a position that is a suitable distance removed from, or is disposed to also engage with, the inner surface of the outer cutting tube 152 in order to cut fibrils or tissue without creating damaging traction on the retina.

At 804, a guiding surface of the inner cutting tube may guide the distal sliding motion of the inner cutting tube 154 as the tip of the inner cutting tube 154 traverses the gap of the outer port in the distal direction. The guiding surface guides the sliding motion, for example, by slidably bearing on an inner surface of the outer cutting tube 152 at a proximal side of the outer port. The guiding motion assists in preventing the tip from protruding too far beyond a diameter of the outer cutting tube 152 as a result of the bend in the inner cutting tube 154.

At 806, the vitrectomy probe 112 aspirates any cut tissue from the distal end of the inner cutting tube 154, for example via the distal port. The aspiration may be via the suction port 162 that connects the vitrectomy probe 112 to an aspiration system on the base housing 102. This may occur, for example, when the inner cutting tube 154 has been fully extended in the distal direction, in the “closed” position such that the inner port 404 and the outer port 408 are at least partially aligned.

At 808, the vitrectomy probe 112 causes the inner cutting tube 154 to slide in the proximal direction. As the cutting edge at the proximal side of the tip moves proximally, the cutting edge cuts tissue that has entered the inner port and the outer port while they are aligned, either substantially or partially.

At 810, the guiding surface guides the proximal sliding motion of the inner cutting tube 154 as the tip 406 traverses the gap of the outer port in the proximal direction. The guiding surface guides the sliding motion, for example, by slidably bearing on an inner surface of the outer cutting tube 152 at a proximal side of the outer port.

At 812, the vitrectomy probe 112 aspirates any cut tissue and/or fluids from the area of the inner port. The aspiration may again be via the suction port 162. In some embodiments, the inner cutter may have a simple inner port 404 without side lobes. In some embodiments, the inner cutter may not have an inner port at all (e.g., tissue may be cut primarily by edge 504 traversing outer port 408). In embodiments with side lobes, at least some of the cut tissue and/or fluids may be aspirated at 812 via one or both of the side lobes. This may facilitate the more efficient removal of vitreous/membranes without excessive flow of balanced saline solution. In some embodiments, the aspiration system may draw a constant or continuous vacuum pressure at the suction port 162 and the bore (lumen) of the inner cutting tube 154.

In embodiments with side lobes 410, the side lobes 410, together, may serve to define the guiding surface 402 as a protrusion in the distal direction from the otherwise generally uniform shape of the inner port 404 into the space of the inner port 404. The guiding surface 402 provides an additional surface which may slidably bear on an inner surface of the outer cutting tube 152 as the inner cutting tube 154 axially moves in the distal and proximal directions during operation.

As a result, in embodiments where the inner cutting tube 154 has been curved more than the outer tube to provide a flexural side load, the guiding surface 402 may remain in contact with the inner surface of the outer cutting tube 152 while the inner cutting tube 154 axially moves in the distal direction. This contact between the guiding surface 402 and the inner surface of the outer cutting tube 152 may prevent the inner cutting tube 154 from protruding too far out of the outer port 408, thereby decreasing the chance of tortuous or impeded cutter motion and reducing wear on the vitrectomy probe 112. The guiding surface 402 may be formed from the rest of the inner cutting tube 154. Alternatively, the guiding surface may be separately formed and secured in place at a proximal side of the inner port 404 using welding, brazing, cements, adhesives, friction fits, or other methods.

In embodiments with side lobes, the side lobes 410 may extend the surface area of the inner port 404, thereby allowing more fluid flow via the inner port 404 than would be available to a port with a conventional contour (e.g., without side lobes) in the inner cutting tube 154. In an embodiment, addition of the side lobes 410 to the vitrectomy probe 112 in embodiments of the present disclosure enables operation of the vitrectomy probe 112 at reduced vacuum settings while still providing equivalent vitreous flow and reduced traction transmitted to the retina relative to conventional vitrectomy probes. In an embodiment, the length of each side lobe 410 along the longitudinal axis of the inner cutting tube 154 is less than a width of the inner port 404 between the tip 406 and the proximal portion where the guiding surface 402 is located. In an alternative embodiment, the length of each side lobe 410 along the longitudinal axis of the inner cutting tube 154 is greater than a width of the inner port 404 between the tip 406 and the proximal portion where the guiding surface 402 is located.

The size of the side lobes 410 may impact how much additional fluid flow is possible above and beyond what is already available were the inner port 404 shaped in a conventional configuration, e.g. in a round or oval shape. This is because the vitreous humor, as well as tractional and vascular membranes that are commonly removed by surgical use of vitrectomy probes, are non-homogenous, non-Newtonian substances that tend to resist flow through small orifices and lumens such as those in conventional vitrectomy probes. Further, balanced saline solution—which is commonly used as an adjunct during vitreoretinal surgery—is a low viscosity Newtonian fluid that is much more able to flow through smaller passages. As a result, aspiration flow in cutter ports of conventional vitrectomy probes may result in a disproportionately high amount of balanced saline solution instead of vitreous/membranes. The larger inner port size afforded by the side lobes 410 in embodiments of the present disclosure may facilitate the more efficient removal of vitreous/membranes without excessive flow of balanced saline solution.

800 through 812 may continue throughout the duration of operation of the vitrectomy probe 112, resulting in cutting cycle of the vitrectomy probe 112 with improved fluid flow and reduced sliding wear on the vitrectomy probe 112 over time. This may result in a longer lasting vitrectomy probe, a smoother cutting cycle, and greater cut rates while decreasing risks of traction on the retina.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure.

Accordingly, it is appropriate that the appended claims be construed broadly and in a manner consistent with the present disclosure.

What is claimed is:

1. A vitrectomy probe, comprising:
a hand-graspable body;
an outer tube extending from the hand-graspable body and sized to penetrate an eye of a patient during an ocular surgery, the outer tube having a closed distal end and a first port sized to receive vitreous of an eye, wherein the outer tube has a proximal portion adjacent to the hand-graspable body and a distal portion opposite the proximal portion; and
an inner tube disposed at least partially within the outer tube;
wherein the outer tube is curved along the distal portion of the outer tube.

2. The vitrectomy probe of claim 1, wherein the inner tube is curved along a distal portion of the inner tube that coincides with the curved distal portion of the outer tube.

3. The vitrectomy probe of claim 1, wherein the outer tube is curved along only 5 to 10 percent of a distal-most length of the outer tube external to the hand-graspable body.

4. The vitrectomy probe of claim 1, wherein the outer tube is curved along only 11 to 20 percent of a distal-most length of the outer tube external to the hand-graspable body.

5. The vitrectomy probe of claim 1, wherein the outer tube is curved along only 21 to 30 percent of a distal-most length of the outer tube external to the hand-graspable body.

6. The vitrectomy probe of claim 1, wherein the outer tube is made of a superelastic material.

7. The vitrectomy probe of claim 1, wherein the inner tube is made of a superelastic material.

8. The vitrectomy probe of claim 1, wherein a curvature of the outer tube is adjustable.

9. The vitrectomy probe of claim 1, wherein the curvature of the outer tube is adjustable through user manipulation of a user actuated slider, lever, or wheel.

10. The vitrectomy probe of claim 1, further comprising a wheel gear turned to engage a toothed shaft to move the shaft up and down inside the outer tube to adjust the curvature of the outer tube.

11. A method of operating a vitrectomy probe, comprising:
adjusting a curvature of an outer tube of the vitrectomy probe; and
moving an inner cutter inside the outer tube to cut tissue extending into a port of the outer tube, wherein the inner cutter moves along a curved path inside the outer tube.

12. The method of claim 11, wherein the curvature of the outer tube is adjusted during surgery.

13. The method of claim 11, wherein adjusting the curvature of the outer tube comprises a user moving a slider, lever, or wheel on the vitrectomy probe.
